(19) United States
(12) Patent Application Publication
(10) Pub. No.: US 2010/0137851 A1
(43) Pub. Date: Jun. 3, 2010

(54) LASER SURGICAL APPARATUS

(75) Inventors: Shih-Ting Lin, Tainan City (TW); Hsin-Chia Su, Yunlin County (TW); Li-Ting Wang, Pingtung County (TW); Hong-Xi Cao, Kaohsiung County (TW); Chieh Hu, Chiayi City (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(73) Assignee: INDUSTRIAL TECHNOLOGY RESEARCH INSTITUTE, Hsinchu (TW)

(21) Appl. No.: 12/356,540

(22) Filed: Jan. 21, 2009

(30) Foreign Application Priority Data
Dec. 1, 2008 (TW) 97146623

(51) Int. Cl. A61B 18/20

(52) U.S. Cl. 606/10

(57) ABSTRACT

A laser surgical apparatus is provided. The laser surgical apparatus includes a laser generator, a laser delivery module for delivering a laser beam to biological tissues, a fluid source, a fluid delivery module for delivering a fluid to the biological tissues, and a control module for controlling all the above units.

### CROSS-REFERENCE TO RELATED APPLICATION

This application claims the priority benefit of Taiwan application serial no. 97146623, filed Dec. 1, 2008. The entirety of the above-mentioned patent application is hereby incorporated by reference herein and made a part of specification.

### BACKGROUND OF THE INVENTION

1. Field of the Invention

The present invention generally relates to a surgical apparatus, in particular, to a mid-infrared laser surgical apparatus.

2. Description of Related Art

In the last decade, the concept of using laser as a surgical knife is widely accepted in the cosmetic surgery and therapeutic field. The advantages include (1) less bleeding, (2) small surgery wound and quick recovery, and (3) low surgery risks of contact infection between patients and doctors or nurses. The operating principle is as follows. The laser is irradiated on biological tissues, and is absorbed by a specific tissue. The light energy of the laser is converted into thermal energy, which is used to vaporize the biological tissue, stop the bleeding, or even dissect the tissue for performing the surgery.

The tissue to be resected or ablated must have high absorption to a laser source of the laser knife. As shown in FIG. 1, the tissue to be resected or ablated has a very high absorption to the ultraviolet (UV) and mid-infrared laser source, so a detailed description is given for this range.

(1) UV Range Laser Source:

This range covers ArF and KrF excimer lasers. The characteristics of small wavelength and high energy of these lasers may cause DNA breakage and protein denaturation in tissues, so the ArF and KrF excimer lasers are unsuitable as therapeutic optical sources for tissue resection.

(2) Near-Infrared (NIR) Range Laser Source:

The technology of neodymium:yttrium-aluminum-garnet (Nd:YAG) laser and semiconductor lasers is mature in the industry for providing high power or pulse output products. However, the lasers in this range are difficult to be absorbed by tissues, and are not suitable as therapeutic optical sources for tissue resection.

(3) Mid-Infrared (Mid-IR) Range Laser Source:

Carbon dioxide (CO₂) laser can be easily absorbed by tissues, but its application is limited by unable being transmitted through optical fibers. On the contrary, the mid-IR lasers may be easily absorbed by tissues, and is applicable to the resection of soft and hard tissues (for example, skin, gingiva, and bones). Further, the mid-IR lasers can be transmitted through optical fibers, and thus become the mainstream therapeutic laser source.

Currently, in the laser therapy, the laser source is often irradiated directly on a target tissue, which may lead to carbonization of the tissue or protein denaturation due to the high energy of the laser source.

One potential approach is to add a cooling procedure to cool the tissue during the laser resection or ablation, so as to solve the above problems of the laser surgery.

### SUMMARY OF THE INVENTION

Accordingly, the present invention is directed to a laser surgical apparatus applicable to biological tissues. The laser surgical apparatus includes a laser generator, a laser delivery module for delivering a laser beam to biological tissues, a high-pressure fluid source, a fluid delivery module for directly delivering a fluid to the biological tissues, and a control module for controlling all the above units.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic view of absorption coefficients of a biological tissue with a water content of approximately 75% to laser sources in different wavelengths.

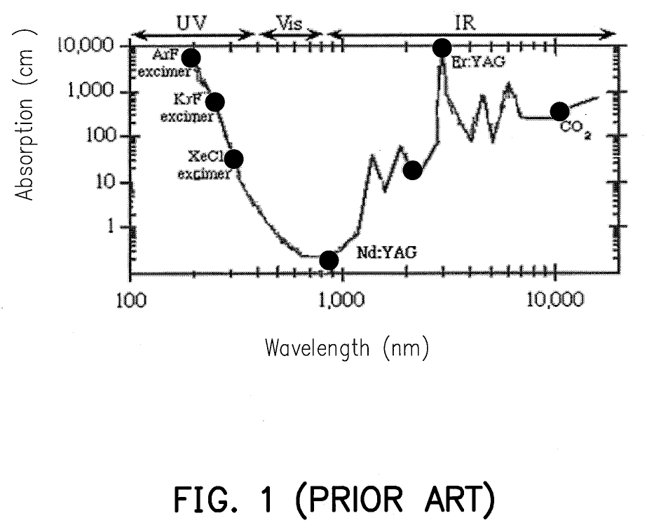

FIG. 2 is a schematic view of a mid-infrared laser surgical apparatus system according to an embodiment of the present invention.

FIG. 3 is a schematic view of a part of the mid-infrared laser surgical apparatus system according to an embodiment of the present invention.

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts.

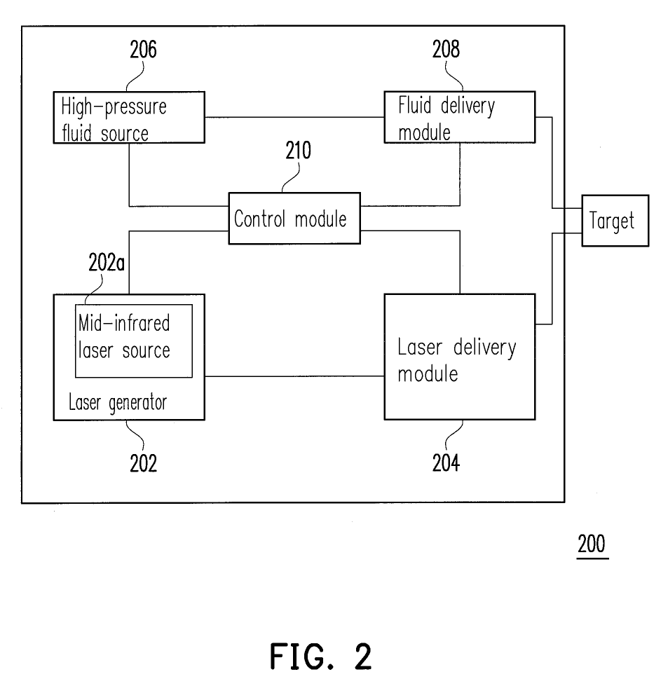

FIG. 2 is a schematic view of a mid-infrared laser surgical apparatus system according to an embodiment of the present invention. The laser surgical apparatus system 200 at least includes a laser generator 202, a laser delivery module 204, a high-pressure fluid source 206, a fluid delivery module 208, and a control module 210. The laser generator 202 is connected to the laser delivery module 204. The laser generator 202 at least includes a mid-infrared laser source 202a for generating a laser beam B, and the laser beam B is then delivered to a target object or target tissue through the laser delivery module. The high-pressure fluid source 206 is connected to the fluid delivery module 208. The high-pressure fluid source 206 at least provides a CO₂ fluid for the fluid delivery module 208. The low-temperature CO₂ fluid is then directly delivered to the target object or the target tissue through the fluid delivery module 208.

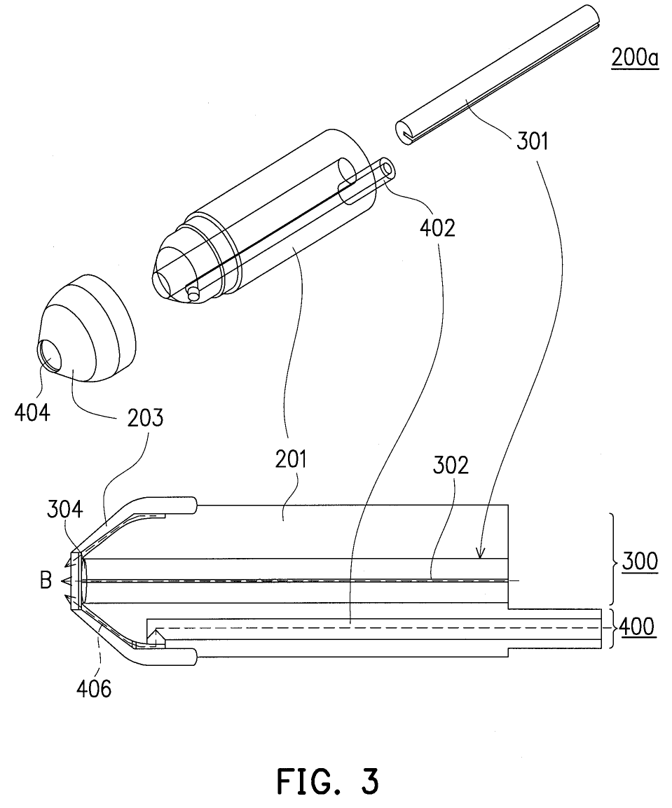

FIG. 3 is a schematic view of a part of the mid-infrared laser surgical apparatus system according to an embodiment of the present invention, in which the upper portion is a partial exploded view for illustrating the specific structure, and the lower portion is its corresponding schematic cross-sectional view. The part shown in FIG. 3 is the terminal part near the target object or target tissue in the infrared laser surgical apparatus system, which is known as a surgical knife or surgical pen. The laser surgical apparatus system 200a may use a tube-shaped or pen-shaped casing 201 to integrate the laser delivery module 300 and the fluid delivery module 400. The laser delivery module 300 at least includes a waveguide or an optical fiber 302 for guiding the laser beam. The waveguide or optical fiber 302 may be fixed or supported by a fixing support 301. The delivery path and direction of the laser beam B is shown as a thick dashed line with arrow respectively. According to the design requirements, the laser delivery module 300 may further include one or more lenses 304. The lenses 304 are fixed to the rear end of the delivery path of the laser beam (that is, near the outlet of the laser beam), for example, by using the casing of the laser delivery module 300 for assisting in focusing the laser beam. The lenses 304 may be fixed through the shape design of the casing 201. Preferably, the lenses 304 are disposed at the end of the optical fiber 302 for assisting in focusing the laser beam on a predetermined position of the target tissue.

The fluid delivery module 400 at least includes a fluid transmission pipe 402 and a fluid nozzle 404. The shape or size of apertures of the nozzle 404 may be adjusted according to the spray distance, the pressure, and spray coverage of the fluid. Generally, the low-temperature CO₂ delivered through the fluid transmission pipe 402 may be directly sprayed out from the fluid nozzle 404 (i.e. the fluid transmission pipe 402 is in direct contact with the nozzle 404). However, in this design, the spray area of the low-temperature CO₂ might be limited by the contact area of the nozzle 404 and the outlet of the transmission pipe 402. According to the design requirements, a sleeve 203 may be further disposed at the rear end of the tube-shaped or pen-shaped casing 201 (that is, near the outlet of the laser beam). Thus, a fluid guiding channel 406 is formed by using an annular gap between the sleeve 203 and the casing 201. The fluid path between the fluid transmission pipe 402 and the nozzle 404 is connected through the fluid guiding channel 406. Thereby, the lower-temperature CO₂ is distributed in the annular fluid guiding channel 406 uniformly, and is then sprayed out from the fluid nozzle 404. As such, the low-temperature CO₂ fluid is sprayed to an area uniformly surrounding the location of the laser beam, and helps to lower the temperature. The delivery path and direction of the low-temperature CO₂ fluid are shown by thin dashed lines with arrows respectively.

In addition, when the present invention is designed as a handheld surgical apparatus, the size of the laser surgical apparatus system is made smaller, and the surgical apparatus can be used more conveniently. At this time, the casing shape may adopt a design that integrates or tightly connects the laser delivery module 300 and the fluid delivery module 400, so as to aim at the target or precisely control the surgery area in a surgery.

In an embodiment of the present invention, the laser beam and the low-temperature CO₂ fluid may be directly delivered to substantially the same position on the target tissue or target object at the same time, so as to burn, resect, or ablate the target tissue. However, according to the target object or application area of the present invention, the low-temperature CO₂ fluid may be delivered to the target tissue or target object first, so as to enhance the anesthetic or cooling effect. Generally, the contact area (i.e., the functioning area) of the low-temperature CO₂ fluid and the target tissue or target object is controlled to be slightly greater than or approximately equal to the contact area (i.e., the functioning area) of the mid-infrared laser and the target tissue or target object.

In FIG. 2, the control module 210 has multiple functions, for example, controlling the laser generator 202 to generate a laser in an appropriate wavelength range or energy density, and controlling the generated mid-infrared laser to be output in a pulse mode or a non-pulse continuous mode to the target tissue or target object.

Further, the control module 210 needs to control the ON/OFF of the high-pressure fluid source 206 and/or the fluid delivery module 208, so as to control the delivery speed of the low-temperature CO₂ fluid. In an embodiment of the present invention, the control module 210 may control the low-temperature CO₂ fluid to be delivered continuously or intermittently when the laser is applied on the target tissue or target object.

The design of the laser surgical apparatus provided in the present invention mainly uses the mid-infrared laser source. Generally, the laser source is a semiconductor laser source in the mid-infrared wavelength range of 2.3 μm to 2.8 μm, preferably in the wavelength range of 2.5 μm to 2.8 μm, and more preferably in the wavelength range of 2.65 μm to 2.75 μm. The laser source in the mid-infrared wavelength range may be any known semiconductor laser source or other laser sources capable of providing an appropriate wavelength range and energy density.

In an embodiment of the present invention, for example, the mid-infrared laser output from the laser surgical apparatus 200 is output in the pulse mode, the interval between pulse signals is preferably 100 μs to 500 ms, the waveform signal width is 10 ps to 500 μs, and the intensity of the output signals is preferably 1 mJ to 100 mJ. [0027] It should be noted that the present invention uses a laser with a wavelength of 2.7 μm, and uses the low-temperature CO₂ liquid as the coolant. The low-temperature CO₂ has acceptable cooling and anesthetic effect on the target tissue. Further, CO₂ has a strong absorption capability to the wavelength of 2.7 μm, and the low-temperature CO₂ can make the air condensed into water drops. Thus, the water and CO₂ will absorb the laser source to form high-energy molecules when the laser is applied, so as to help ablate the tissue and improve the performance of the laser surgical knife. In addition, as the high absorption capability of CO₂ increases the temperature of CO₂, potential damages to the tissue due to excessively low temperature of CO₂ can be avoided.

In the mid-infrared surgical apparatus of the present invention, as CO₂ and water have good absorption capability to the 2.7 μm laser source, the present invention employs the laser source with the wavelength of 2.7 μm and the low-temperature liquid CO₂ to achieve purposes of cooling down. The low-temperature liquid CO₂ can condense the vapour in the air, and CO₂ and the condensed water have good absorption capability to the 2.7 μm laser source. After absorbing the energy of the 2.7 μm laser source, the volume of the liquid CO₂ and water will be expanded by several hundreds of times to form high-energy CO₂ and water molecules, thus helping ablating the tissue. Moreover, the low-temperature CO₂ has a cooling effect, which helps to enhance the anesthetic effect of the tissue and reduce the protein denaturation in the surrounding tissue caused by heat.

The mid-infrared laser surgical apparatus of the present invention is quite practical in the ablation or resection of skin tissues, and is especially suitable for the fields of general therapeutic treatments, cosmetics surgery and dentosurgery.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A laser surgical apparatus, comprising:
a laser generator, comprising a mid-infrared laser source for generating a laser beam;
a laser delivery module, connected to the laser generator, wherein the laser beam is delivered to a biological tissue through the laser delivery module;
a high-pressure fluid source, for providing a low-temperature CO₂ fluid;
a fluid delivery module, connected to the high-pressure fluid source, for delivering the low-temperature CO₂ fluid directly to the biological tissue; and
a control module, electrically connected to and controlling the laser generator, the laser delivery module, the high-pressure fluid source, and the fluid delivery module.

2. The laser surgical apparatus according to claim 1, wherein the laser delivery module further comprises a waveguide or an optical fiber for guiding the laser beam.

3. The laser surgical apparatus according to claim 1, wherein the laser delivery module further comprises one or more lenses for helping focus the laser beam.

4. The laser surgical apparatus according to claim 1, wherein the fluid delivery module at least comprises a fluid transmission pipe and a fluid nozzle connected to the fluid transmission pipe, and the low-temperature CO₂ fluid is sprayed out from the fluid nozzle.

5. The laser surgical apparatus according to claim 1, wherein the control module controls to output the low-temperature CO₂ fluid continuously.

6. The laser surgical apparatus according to claim 1, wherein the control module controls to output and deliver the low-temperature CO₂ fluid and the laser beam to the biological tissue at the same time.

7. The laser surgical apparatus according to claim 1, wherein the control module controls to spray out the low-temperature CO₂ fluid first, and then to deliver the laser beam to the biological tissue.

8. The laser surgical apparatus according to claim 1, wherein the control module controls to output the generated laser beam in a pulse mode.

9. The laser surgical apparatus according to claim 8, wherein the control module controls that an intensity of each laser pulse output signal of the generated laser beam is from 1 mJ to 100 mJ.

10. The laser surgical apparatus according to claim 9, wherein an interval of each laser pulse output signal is from 100 μs to 500 ms.

11. The laser surgical apparatus according to claim 9, wherein a waveform signal width of each laser pulse output signal is from 10 ps to 500 μs.

12. The laser surgical apparatus according to claim 1, wherein the control module controls to output the generated laser beam in a non-pulse continuous mode.

13. The laser surgical apparatus according to claim 1, wherein a wavelength of the mid-infrared laser source is from 2.5 μm to 2.8 μm.

14. The laser surgical apparatus according to claim 13, wherein a wavelength of the mid-infrared laser source is from 2.65 μm to 2.75 μm.

15. The laser surgical apparatus according to claim 1, wherein the control module controls to deliver the CO₂ and the laser beam to the same position on the biological tissue.
